(19) United States
(12) Patent Application Publication
(10) Pub. No.: US 2021/0287793 A1
(43) Pub. Date: Sep. 16, 2021

(54) MEDICAL SYSTEM AND CONTROL METHOD THEREOF

(71) Applicant: HTC Corporation, Taoyuan City (TW)

(72) Inventors: Yang-En CHEN, Taoyuan City (TW); Kai-Fu TANG, Taoyuan City (TW); Edward CHANG, Taoyuan City (TW)

(21) Appl. No.: 17/198,274

(22) Filed: Mar. 11, 2021

Related U.S. Application Data
(60) Provisional application No. 62/987,881, filed on Mar. 11, 2020.

(51) Int. Cl. G16H 40/63; G06N 3/02; G06F 16/903; G16H 10/60

(52) U.S. Cl. CPC G16H 40/63; G16H 10/60; G06F 16/90335; G06N 3/02

(57) ABSTRACT

A control method includes following operations. A symptom input status and a test result status are collected. A neural network is utilized to generate a test suggestion, a predicted test result distribution and a predicted disease distribution according to the symptom input status and the test result status. The test suggestion includes a candidate test. Information gains of the candidate test relative to diseases are estimated according to the predicted test result distribution and the predicted disease distribution. An explainable description about the test suggestion is generated according to the information gains of the candidate test. Another explainable description about a predicted disease list can be generated according to an attention input.

### RELATED APPLICATIONS

This application claims priority to U.S. Provisional Application Ser. No. 62/987,881, filed Mar. 11, 2020, which is herein incorporated by reference.

### BACKGROUND

### Field of Invention

The disclosure relates to a medical system for generating a medical suggestion. More particularly, the disclosure relates to an AI-based medical system capable of providing explainable descriptions about the medical suggestion.

### Description of Related Art

Recently the concept of computer-aided medical system has emerged in order to facilitate diagnosis for patients. The computer-aided medical system may request patients to provide some information, and then the computer aided medical system may provide a diagnosis or a recommendation of the potential diseases based on the interactions with those patients. The computer-aided medical system may aid a doctor in diagnosing, or aid a patient in consulting or self-diagnosing.

Most of the computer-aided medical system utilizes an Artificial Intelligence technology (including machine learning and/or neural network model) to predict the potential diseases or give related recommendations. However, the AI-based technology usually provides a result (diagnosis or recommendation) without any explanation. Therefore, it is hard for the patient or the doctor to trust or to understand the result provided by the AI-based technology.

### SUMMARY

The disclosure provides a control method which includes following operations. A symptom input status and a test result status are collected. A neural network is utilized to generate a test suggestion, a predicted test result distribution and a predicted disease distribution according to the symptom input status and the test result status. The test suggestion includes a candidate test. Information gains of the candidate test relative to diseases are estimated according to the predicted test result distribution and the predicted disease distribution. An explainable description about the test suggestion is generated according to the information gains of the candidate test.

The disclosure provides a control method which includes following operations. A symptom input status and a test result status are collected. The symptom input status includes symptom answers. The test result status includes test results. A neural network is utilized to generate a predicted disease distribution according to the symptom input status and the test result status. A predicted disease list is generated according to the predicted disease distribution. An attention mask is applied to filter the symptom answers and the test results for obtaining an attention input. An explainable description about the predicted disease list is generated according to the attention input.

The disclosure provides a medical system, which includes an interface and a processor. The interface is configured for receiving a symptom input status and a test result status. The symptom input status includes symptom answers. The test result status includes test results. The processor is coupled with the interface.

According to some embodiments, in a test suggestion phase, the processor utilizes a neural network to generate a test suggestion, a predicted test result distribution and a predicted disease distribution according to the symptom input status and the test result status. The test suggestion includes a candidate test. The processor estimates a plurality of information gains of the candidate test relative to diseases according to the predicted test result distribution and the predicted disease distribution. The processor generates a first explainable description about the test suggestion according to the information gains of the candidate test.

According to some embodiments, in a disease prediction phase, the processor generates a predicted disease list according to the predicted disease distribution. The processor applies an attention mask to filter the symptom answers and the test results for obtaining an attention input. The processor generates a second explainable description about the predicted disease list according to the attention input.

In some embodiments, the disclosure can provide AI decision-making explanations for the automatic diagnosis system in the test suggestion phase and the disease prediction phase. In the test suggestion phase, the distribution of test results predicted by the neural network can be used to calculate the information gain of each test in distinguishing different diseases, so as to explain a correspondence between the suggest test and a target disease to distinguish. In the disease prediction phase, an attention mask is utilized to find out critical symptoms and/or critical test results, which are important for disease prediction.

It is to be understood that both the foregoing general description and the following detailed description are demonstrated by examples, and are intended to provide further explanation of the invention as claimed.

### BRIEF DESCRIPTION OF THE DRAWINGS

Embodiments of the invention will now be described with reference to the attached drawings in which:

FIG. 1 is a schematic diagram illustrating a medical system according to some embodiments of the disclosure.

FIG. 2 is a schematic diagram illustrating functional blocks of the processor in FIG. 1 according to some embodiments of the disclosure.

FIG. 3 is a flow chart illustrating a control method for controlling the medical system in FIG. 1 according to some embodiments of the disclosure.

FIG. 4, which is a schematic diagram about a symptom input status and a test result status of an input status according to some embodiments.

FIG. 5, which is a schematic diagram about symptom-query state values, test-suggestion state values, a predicted disease distribution Odd and a predicted test result distribution in the output status according to some embodiments.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

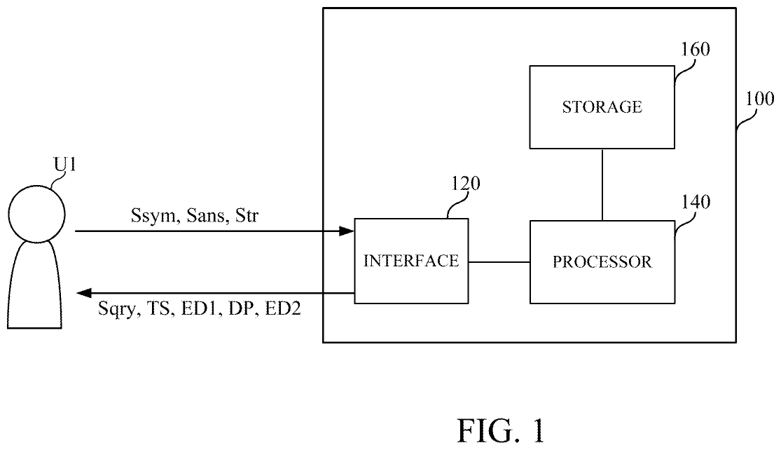

Reference is made to FIG. 1, which is a schematic diagram illustrating a medical system 100 according to some embodiments of the disclosure. As depicted in FIG. 1, the medical system 100 includes an interface 120, a processor 140 and a storage 160.

In some embodiments, the processor 140 is communicated with the interface 120. The medical system 100 is configured to interact with the user U1 through the interface 120 (e.g. collecting a symptom input status Ssym from the user U1, providing some symptom inquiries Sqry to the user U1, collecting corresponding symptom responses Sans from the user U1, providing a test suggestion TS to the user U1, collecting test result inputs Str from the user U1). Based on aforesaid interaction history, the medical system 100 is able to analyze, diagnose or predict a potential disease occurring to the user U1.

The medical system 100 is trained with a machine learning algorithm or a reinforcement learning algorithm, such that the medical system 100 is capable to inquire and diagnose based on limited patient data. In some embodiments, the medical system 100 adopts a reinforcement learning (RL) framework to formulate inquiry and diagnosis policies (e.g., Markov decision processes). In some embodiments, a neural network is trained based on the machine learning algorithm or the reinforcement learning algorithm by the processor 140 according to some training data (e.g., known medical records) and the trained neural network is stored in the storage 160.

In some embodiments, the medical system 100 is established with a computer, a server or a processing center. The processor 140 can be implemented by a central processing unit (CPU), a graphic processing unit (GPU), a tensor processing unit (TPU), an application-specific integrated circuit (ASIC) or any equivalent computation unit. The interface 120 can include an output interface (e.g., a display panel for display information) and an input device (e.g., a touch panel, a keyboard, a microphone, a scanner or a flash memory reader) for user to type text commands, to give voice commands or to upload some related data (e.g., images, medical records, or personal examination reports). As shown in FIG. 1, the storage 160 is coupled with the processor 140. In some embodiments, the storage unit 160 can be implemented by a memory, a flash memory, a ROM, a hard drive or any equivalent storage component.

As shown in FIG. 1, the interface 120 can be manipulated by a user U1. The user U1 can see the information displayed on the interface 120 and the user U1 can enter his/her inputs on the interface 120. In an embodiment, the interface 120 will display a notification to ask the user U1 about his/her symptoms. The interface 120 is configured for collecting a symptom input status Ssym about the user U1. The interface 120 may also collects other information about the user U1. For example, a medical test (e.g., a blood pressure test, a rapid influenza diagnostic test) is already performed on the user U1, and a test result status Str can be collected by the interface 120. The interface 120 transmits the symptom input status Ssym and the test result status Str to the processor 140.

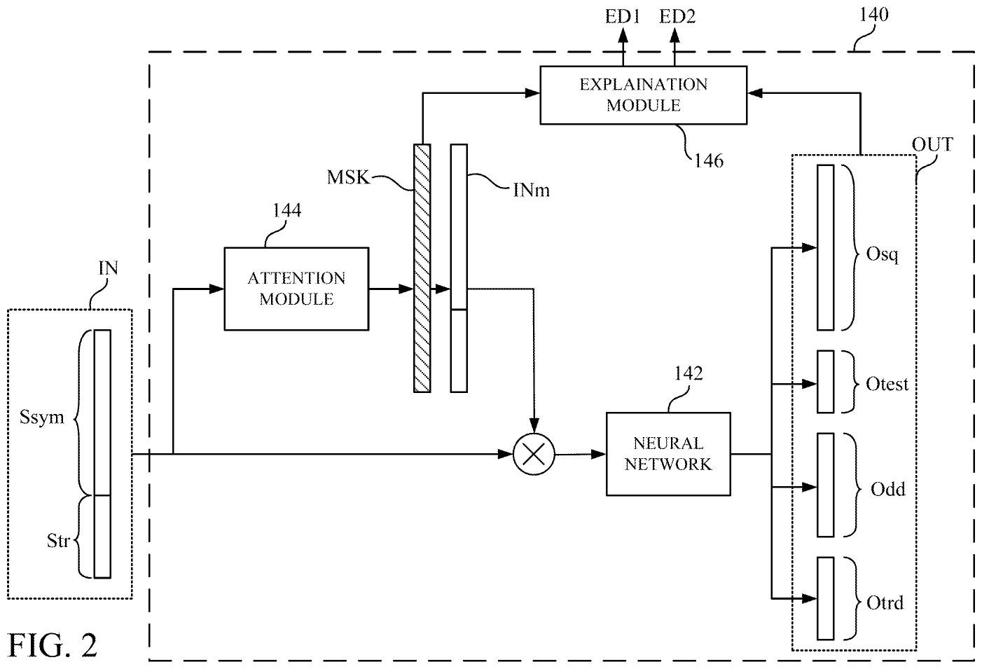

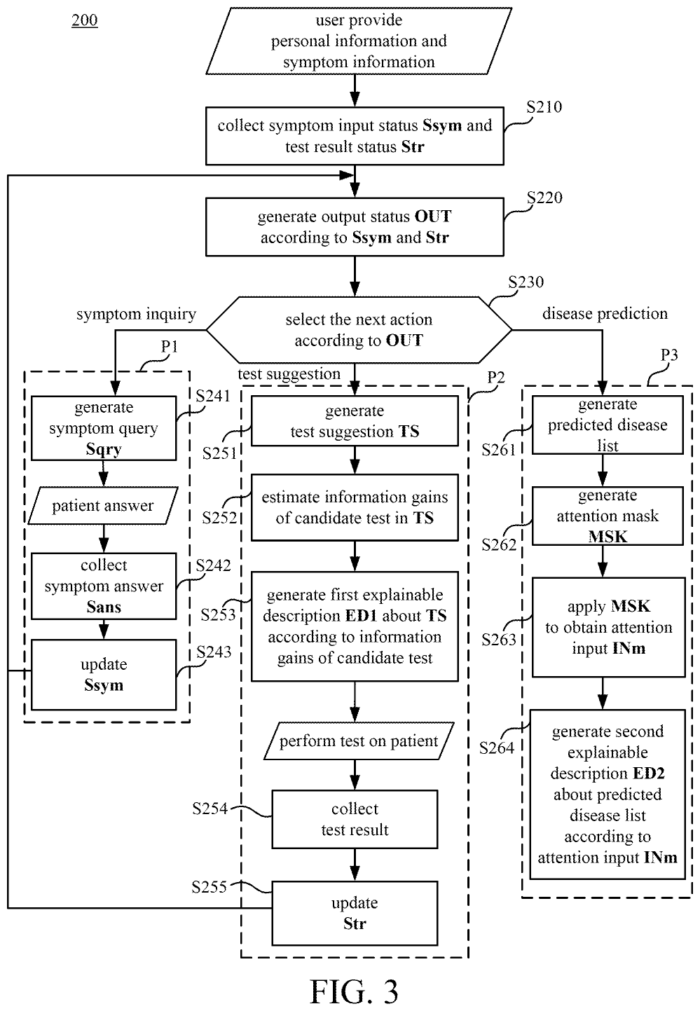

Reference is further made to FIG. 2 and FIG. 3. FIG. 2 is a schematic diagram illustrating functional blocks of the processor 140 in FIG. 1 according to some embodiments of the disclosure. FIG. 3 is a flow chart illustrating a control method 200 for controlling the medical system 100 in FIG. 1 according to some embodiments of the disclosure.

As shown in FIG. 1, FIG. 2 and FIG. 3, when the user U1 provides personal information (e.g., whether a medical test result on the user U1 is positive or negative) and symptom information (e.g., whether the user U1 suffers some symptoms, such as fever, cough, headache), in step S210, the interfaces 120 can collects the input status IN (including the symptom input status Ssym and the test result status Str), and transmits the input status IN to the processor 140. In step S220, the processor 140 receives input status IN (including the symptom input status Ssym and the test result status Str), the processor 140 utilize a neural network 142 to generate symptom-query state values Osq, test-suggestion state values Otest, a predicted disease distribution Odd and a predicted test result distribution Otrd according to the symptom input status Ssym and the test result status Str.

In some embodiments, the neural network 142 can be trained with a machine learning algorithm or a reinforcement learning algorithm according to the training data in advance. In some embodiments, the training data includes known medical records. The medical system 100 utilizes the known medical records in the training data to train the neural network 142. In an example, the training data can be obtained from data and statistics information from the Centers for Disease Control and Prevention (www.cdc.gov/datastatistics/index.html). The medical system 100 is capable to inquire and diagnose based on limited patient data. Further details about how to train the neural network 142 will be discussed later in some other embodiments.

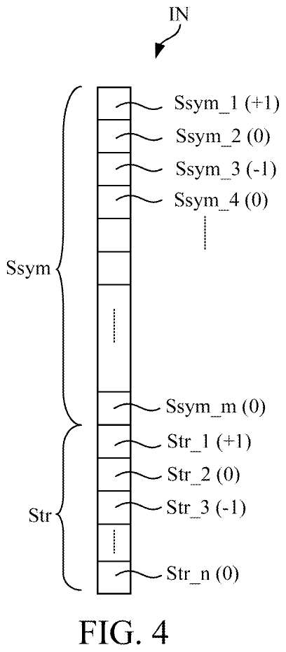

After training, the neural network 142 is able to generate output status OUT based on contents of the symptom input status Ssym and the test result status Str of the input status IN. Reference is further made to FIG. 4, which is a schematic diagram about the symptom input status Ssym and the test result status Str of the input status IN according to some embodiments.

As shown in FIG. 4, the symptom input status Ssym includes “m” data digits Ssym_1, Ssym_2, Ssym_3, Ssym_4 . . . and Ssym_m. It is noticed that, m is a positive integer corresponding to a total amount of symptoms considered by the medical system 100. Each of the data bits Ssym_1 to Ssym_m indicates whether the user U1 has one corresponding symptom. For example, the data digit Ssym_1 is set to “1” indicating that the user U1 has a symptom “cough”; the data digit Ssym_3 is set to “−1” indicating that the user U1 does not has another symptom “headache”; the data digit Ssym_2 is set to “0” indicating that it is current unknown whether the user U1 has a symptom “stomach pain” or not; the data digit Ssym_4 is set to “0” indicating that it is current unknown whether the user U1 has a symptom “no appetite” or not.

As shown in FIG. 4, the test result status Str includes “n” data digits Str_1, Str_2, Str_3 . . . and Str_n. Each of the data digits Str_1 to Str_n indicates a test result about one medical test performed on the user U1. It is noticed that, n is a positive integer corresponding to a total amount of medical tests considered by the medical system 100. For example, the data digit Str_1 is set to “1” indicating that a test result is “positive” about the first medical test; the data digit Str_3 is set to “−1” indicating that a test result is “negative” about the third medical test; the data digit Ssym_2 is set to “0” indicating that the second test is not performed on the user U1 yet. Aforesaid definitions of digits are examples for demonstration, and the disclosure is not limited thereto.

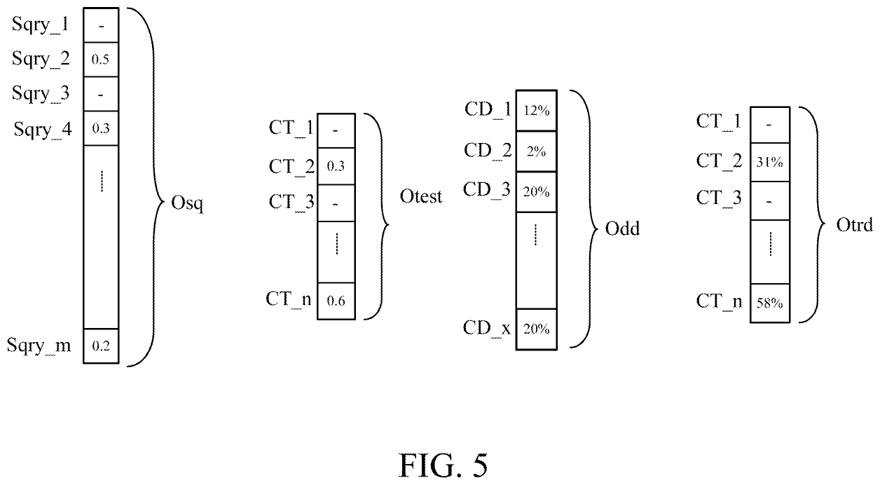

In step S220, the neural network 142 is able to generate output status OUT based on contents of the symptom input status Ssym and the test result status Str. In some embodiments, the output status OUT includes the symptom-query state values Osq, the test-suggestion state values Otest, the predicted disease distribution Odd and the predicted test result distribution Otrd. Reference is further made to FIG. 5, which is a schematic diagram about the symptom-query state values Osq, the test-suggestion state values Otest, the predicted disease distribution Odd and the predicted test result distribution Otrd in the output status OUT according to some embodiments.

As shown in FIG. 5, the symptom-query state values Osq includes state values corresponding to different symptom queries Sqry_1 to Sqry_m. The test-suggestion state values Otest includes state values corresponding to different candidate tests CT_1 to CT_n. The predicted disease distribution Odd includes probabilities (estimated by the neural network 142 according to the input status IN) of the user U1 has corresponding candidate diseases CD_1 to CD_x. It is noticed that, x is a positive integer corresponding to a total amount of diseases considered by the medical system 100. The predicted test result distribution Otrd includes probabilities (estimated by the neural network 142 according to the input information IN) of the user U1 for getting a target result (e.g., tested positive) in corresponding candidate tests CT_1 to CT_n.

In step S230, the processor 140 select the next action according to the output status OUT. In some embodiments, if the output status OUT indicates the symptom information is not enough (e.g., an amount of the answered symptom queries is less than a threshold, test-suggestion state values Otest is not high enough, or a maximum probability of one candidate diseases CD_1 to CD_x in the predicted disease distribution Odd is not high enough) to give a test suggestion or make a disease prediction, the control method will enter a symptom inquiry phase P1 to generate a symptom query Sqry in step S241. In some embodiments, the symptom query Sqry is generated according to a maximum state value among the symptom-query state values Osq. For example, if the symptom-query state values Osq indicating the symptom query Sqry_2 (e.g., “do you feel pain at your stomach?”) with the maximum state value, the symptom query Sqry_2 is generated and presented to the user U1. The user can answer to the symptom query Sqry_2. In step S242, the symptom answer Sans relative to the symptom query Sqry_2 can be collected from the user U1 through the interface 120. The processor 140 can updates the symptom input status Ssym according to the symptom answer Sans. As shown in FIG. 4, the data digit Ssym_2 will be updated into “+1” if the user U1 has the symptom “stomach pain”, or updated into “−1” if the user U1 does not feel the symptom “stomach pain”. The symptom inquiry phase P1 can be repeated several time, as shown in FIG. 3, until the medical system 100 and the control method 200 gather enough symptom information.

In some embodiments, when the medical system 100 and the control method 200 gather enough symptom information, the medical system 100 and the control method 200 will enter a test suggestion phase P2 as shown in FIG. 3. According to some embodiments, in step S251, the test suggestion TS is generated according to the test-suggestion state values Otest. The test suggestion TS may include one or more medical test(s) with the highest state value(s) among the test-suggestion state values Otest. The following Table 1 shows an example of the test suggestion TS. In the example shown in the following Table 1, the test suggestion TS includes three different medical tests for demonstration, and the disclosure is not limited thereto.

TABLE 1
Suggested Test (TS)

Rapid Influenza diagnostic test (CT1)
Throatology Examination (CT2)
Thoracic Examination (CT3)

In some embodiments, the test suggestion TS includes at least one candidate test(s). As the embodiment shown in Table 1, the test suggestion TS includes three candidate tests CT1˜CT3, which includes Rapid Influenza diagnostic test (CT1), the Throatology Examination (CT2) and the Thoracic Examination (CT3). These candidate tests CT1˜CT3 are suggested according to three highest state values among the test-suggestion state values Otest. The embodiment shown in Table 1, these three candidate tests CT1˜CT3 in the test suggestion TS are illustrated for demonstration, and the medical system 100 and the control method 200 are not limited to provide a specific amount of candidate test(s). For example, the medical system 100 and the control method 200 in some embodiments can provide one candidate test to ten candidate tests in the test suggestion TS. In some AI systems, a test suggestion similar to Table 1 can be generated and provided to the user, but if the test suggestion TS is provided to the user U1 without any explanations, it is hard for the user to understand, to verify, or to trust the test suggestion TS. Sometimes, even a medical profession is hard to explain the test suggestion TS generated by an AI system. In some embodiments discussed in following paragraphs, the medical system 100 and the control method 200 are able to provide a first explainable description ED1 to explain the test suggestion TS.

In step S252, an explanation module 146 in the processor 140 is configured to estimate information gains of each candidate test relative to different diseases according to the predicted test result distribution Otrd and the predicted disease distribution Odd. For example, if there are 70 diseases (D1˜D70) considered in the medical system 100, the explanation module 146 will estimate one information gain between “CT1” and “D1”, another information gain between “CT1” and “D2” . . . and another information gain between “CT1” and “D70”. Similarly, the explanation module 146 will estimate one information gain between “CT2” and “D1”, another information gain between “CT2” and “D2” . . . and another information gain between “CT2” and “D70”. In some embodiments, the explanation module 146 can be implemented by software instructions executed by the processor 140.

For brevity, the following embodiment discussed how to calculating one information gain between one candidate test and one target disease. An information gain Ĝ_{d}(IN, CT) of the candidate test CT on a patient with the current input status IN relative to a target disease “d” can be estimated as below:

Ĝ_{d}(IN,CT)=Î_{d}(IN)−Σ_{vϵV}_{_{CT}}\hat{p}ₜₑₛₜ(IN,CT,v)Î_{d}(IN,CT=v) (1)

The information gain Ĝ_{d}(IN, CT) of the candidate test CT on a patient with the current input status IN relative to a target disease “d” is estimated in reference with a first Gini index Î_{d}(IN) about the target disease “d” in a group with the current input status IN before performing the candidate test CT, a probability \hat{p}ₜₑₛₜ(IN, CT, v) to get a target result “v” in the candidate test CT, and a second Gini index Î_{d}(IN, CT=v) about the target disease “d” in a group with the target result “v” after performing the candidate test CT. The target result “v” is one of possible results V_{CT} of performing the candidate test CT.

In the equation (1) above, the probability \hat{p}ₜₑₛₜ (IN, CT, v) to get a target result “v” in the candidate test CT can be acknowledged according to the predicted test result distribution Otrd generated by the neural network 142.

In aforesaid equation (1), the first Gini index Î_{d}(IN) can be estimated as below:

Î_{d}(IN)=1−π_{dis}(IN,d)²−(1−π_{dis}(IN,d))² (2)

In aforesaid equation (2), π_{dis}(IN, d) is an estimated probability that a patient with the current input status IN is diagnosed to have the target disease “d”. The π_{dis}(IN, d) can be acknowledged according to the predicted disease distribution Odd generated by the neural network 142.

In aforesaid equation (1), the second Gini index Î_{d}(IN, CT=v) can be estimated as below:

Î_{d}(IN,CT=v)=1−π_{dis}(INᵥ,d)²−(1−π_{dis}(IN,d))² (3)

In aforesaid equation (3), π_{dis}(INᵥ, d) is an estimated probability that a patient (with the current input status IN and expected to have the target result “v” in the candidate test CT) is diagnosed to have the target disease “d”. The π_{dis}(INᵥ, d) can be acknowledged by updating the input status IN into another input status INᵥ (filling the target result “v” into the result of the candidate test CT) and utilize the neural network 142 to re-calculate the predicted disease distribution Odd according to the input status INᵥ.

When the information gain Ĝ_{d}(IN, CT) of the candidate test CT is higher, it means the candidate test CT has a higher significance in distinguishing whether the patient with the current input status IN has the target disease “d” or not. When the information gain Ĝ_{d}(IN, CT) of the candidate test CT is lower, it means the candidate test CT is not helpful in distinguishing whether the patient with the current input status IN has the target disease “d” or not. By calculating the information gains Ĝ_{d}(IN, CT) of the candidate test CT on each one of all candidate diseases, the medical system 100 and the control method 200 can acknowledge that the candidate test CT is important in distinguishing which one among all candidate diseases.

In step S253, the explanation module 146 is configured to generate a first explainable description ED1 about the test suggestion TS according to the information gains of the candidate test(s).

The following Table 2 is an example about a first explainable description ED1, which is generated by the medical system 100 and the control method 200 to explain the test suggestion TS.

TABLE 2
Al decision interpretation (ED1)

| Suspected disease | Confidence Level |
|---|---|
| Influenza type A | 26% |
| Influenza type B | 25% |
| Upper respiratory tract infection | 22% |
| Bronchitis | 13% |
| Candidate test | Target distinguish disease |
| Rapid Influenza diagnostic test | Influenza type A |
| | Influenza type B |
| | Upper respiratory tract infection |
| Throatology Examination | Upper respiratory tract infection |
| | Bronchitis |
| Thoracic Examination | Influenza type B |
| | Upper respiratory tract infection |
| | Bronchitis |

As shown in Table 2, the first explainable description ED1 is helpful for the user U1 to understand the test suggestion TS given by the medical system 100 and the control method 200. In some embodiments, the confidence levels of the suspected disease shown in the first explainable description ED1 can be determined by the predicted disease distribution Odd according to the current input status IN. In some embodiments, target distinguish diseases of the candidate tests shown in the first explainable description ED1 can be determined by the information gains of each candidate test relative to each disease.

After the suggest tests are performed on the user U1, the test results about these suggested tests can be collected through the interface 120 in step S254. In step S255, the processor 140 is able to update the test result status Str in the input status IN according to these collected test results. The control method 200 will return to S220, to re-generate the output status OUT by the neural network 142 according to the input status IN after updating. In the case, the predicted disease distribution Odd in the output status OUT will also consider the test results in these suggested tests.

After the symptom inquiry phase P1, the test suggestion phase P2, the medical system 100 and the control method 200 may gather enough information (about symptom queries and test results) to predict a disease of the user U1. The control method 200 will enter a disease prediction phase P3, and in Step S261, the processor 140 generates a predicted disease list DP (and/or a medical department recommendation corresponding to the predicted disease list DP correspondingly) according to the predicted disease distribution Odd generated by the neural network 142 according to the input status IN. As shown in FIG. 1, the predicted disease list DP can be displayed to the user U1 through the interface 120.

The following Table 3 shows an example of the predicted disease list DP. In the example shown in the following Table 3, the predicted disease list DP includes three different diseases for demonstration, and the disclosure is not limited thereto.

TABLE 3

| Predicted diseases | Confidence Level |
|---|---|
| Acute gastroenteritis | 81% |
| Norovirus infection | 12% |
| Cholera | 2% |

In some embodiments, the predicted disease list DP includes at least one predicted disease(s). As shown in Table 3, the predicted disease list DP includes three predicted diseases, which includes Acute gastroenteritis, Norovirus infection and Cholera. These predicted diseases are suggested according to three highest state values among the predicted disease distribution Odd. The embodiment shown in Table 3, these predicted diseases in the predicted disease list DP are illustrated for demonstration, and the medical system 100 and the control method 200 are not limited to provide a specific amount of predicted disease(s). For example, the medical system 100 and the control method 200 in some embodiments can provide one predicted disease to ten predicted diseases in the predicted disease list DP.

In some other embodiments, in Step S261, the processor 140 can generate a medical department recommendation according to the predicted disease list DP, and the medical system 100 and the control method 200 can provide the medical department recommendation to the user U1 instead of the predicted disease list DP. For example, the medical department recommendation may include “Emergency Department” corresponding to the predicted disease “acute gastroenteritis”.

In some AI systems, a test suggestion similar to Table 3 can be generated and provided to the user, but if the predicted disease list DP is provided to the user U1 without any explanations, it is hard for the user to understand, to verify, or to trust the predicted disease list DP. Sometimes, even a medical profession is hard to explain the predicted disease list DP generated by an AI system. In some embodiments discussed in following paragraphs, the medical system 100 and the control method 200 are able to provide a second explainable description ED2 to explain the predicted disease list DP.

In a demonstrational example, information in the input status IN (the symptom input status Ssym and the test result status Str) corresponding to the predicted disease list DP shown in Table 3 may include:

(feature 1) Have fever;

(feature 2) No trauma;

(feature 3) Have cough symptoms;

(feature 4) No symptoms of runny nose;

(feature 5) Symptom of vomiting;

(feature 6) Symptom of diarrhea;

(feature 7) No symptom of general muscle ache; and

(feature 8) Vibrio cholerae culturing test: negative.

In Step S262, referring to FIG. 2 and FIG. 3, an attention mask MSK is generated by an attention module 144 according to the input status IN (including the symptom input status Ssym and the test result status Str). The attention mask MSK is configured to filter the input status IN, by blocking a part of input features and allowing another part of input features to pass. The input features passing through the attention mask MSK is regarded as an attention input INm. Based on different values in the input status IN, the attention mask MSK will be generated by the attention module 144 differently. In some embodiments, the attention module 144 can be implemented by software instructions executed by the processor 140.

In step S263, the attention mask MSK is applied to the input status IN to obtain the attention input INm. For example, the attention mask MSK can allow the features 1, 6 and 7 to pass, such that the attention input INm includes (feature 1) fever; (feature 6) diarrhea and (feature 7) no general muscle ache.

In step S264, the explanation module 146 is configured to generate a second explainable description ED2 about the predicted disease list DP according to the attention input INm.

The following Table 4 is an example about a second explainable description ED2, which is generated by the medical system 100 and the control method 200 to explain the predicted disease list DP in Table 3.

TABLE 4
Al decision interpretation (ED2)

| Prediction base | Importance |
|---|---|
| Symptom of diarrhea | ••• |
| Have fever | ••• |
| No symptom of general muscle ache | •• |

As shown in Table 4, the second explainable description ED2 is helpful for the user U1 to understand the predicted disease list DP given by the medical system 100 and the control method 200.

In some embodiments, the attention module 144 is trained (about how to generate the attention mask MSK according to the input status IN) in a training phase according to some training data. This attention module 144 can be implemented by several fully-connected layers, which will generate a set of attention masks based on the input status IN (can be feed according to the known medical records in the training data), selectively block some of the input features, so that the following neural network 142 will only perform calculations based on some of the features. After training, the neural network 142 can determine which features are important according to the input, and the attention module 144 generates the corresponding attention mask to retain the important features in the input and block the unimportant features, so that the entire model can still generate the correct features based on the filtered input status. By utilizing the attention mask MSK, when the predicted disease list DP is generated, the medical system 100 can identify which input features the predicted disease list DP is based on, according to the attention mask MSK generated by the attention module 144.

In some embodiments, the neural network 142 is trained in advance according to some training data (e.g., known medical records). The processor 140 utilizes the neural network 142 to generate the output status OUT and accordingly selects the sequential actions from a set of candidate actions. In some embodiments, the sequential actions include some symptom inquiry actions, one or more medical test actions (suitable for providing extra information for predicting or diagnosing the disease) and a disease prediction action.

When the processor 140 selects proper actions (e.g., some proper symptom inquiries, some proper medical test actions or a correct disease prediction action matching with the medical records in the training data), corresponding rewards will be provided to the neural network 142. In some embodiments, the neural network 142 is trained to maximize cumulative rewards in response to the sequential actions. In some embodiments, the cumulative rewards can be calculated by a sum of a symptom abnormality reward, a test abnormality reward, a test cost penalty and/or a positive/negative prediction reward. In other words, the neural network model 142 is trained to ask proper symptom inquiries, suggest proper medical tests and make the correct disease prediction at its best.

Based on embodiments above, the disclosure can provide AI decision-making explanations for the automatic diagnosis system in the test suggestion phase and the disease prediction phase. In the test suggestion phase, the distribution of test results predicted by the neural network can be used to calculate the information gain of each test in distinguishing different diseases, so as to explain a correspondence between the suggest test and a target disease to distinguish. In the disease prediction phase, an attention mask is utilized to find out critical symptoms and/or critical test results, which are important for disease prediction.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A control method, comprising:
collecting a symptom input status and a test result status;
utilizing a neural network to generate a test suggestion, a predicted test result distribution and a predicted disease distribution according to the symptom input status and the test result status, wherein the test suggestion comprising a candidate test;
estimating a plurality of information gains of the candidate test relative to a plurality of diseases according to the predicted test result distribution and the predicted disease distribution; and
generating an explainable description about the test suggestion according to the information gains of the candidate test.

2. The control method as claimed in claim 1, wherein the explainable description corresponds to a disease list that the candidate test is capable to distinguish according to the information gains.

3. The control method as claimed in claim 1, wherein one information gain of the candidate test relative to a target disease is estimated in reference with a first Gini index about the target disease in a group before performing the candidate test, a probability to get a target result in the candidate test, and a second Gini index about the target disease in a group with the target result after performing the candidate test.

4. The control method as claimed in claim 3, wherein the first Gini index is obtained according to the predicted disease distribution generated by the neural network under a condition that a result of the candidate test is unknown.

5. The control method as claimed in claim 3, wherein the probability to get the target result in the candidate test is obtained according to the predicted test result distribution.

6. The control method as claimed in claim 3, wherein the second Gini index is obtained according to the predicted disease distribution generated by the neural network under a condition that a result of the candidate test is the target result.

7. The control method as claimed in claim 1, wherein the neural network is trained to generate the test suggestion, the predicted test result distribution and the predicted disease distribution in reference with known medical records.

8. The control method as claimed in claim 1, further comprising:
utilizing the neural network to generate a symptom query;
collecting a symptom answer corresponding to the symptom query; and
updating the symptom input status according to the symptom answer.

9. The control method as claimed in claim 1, wherein the symptom input status comprising a plurality of symptom answers, the test result status comprising a plurality of test results, the control method further comprises:
generate a predicted disease list according to the predicted disease distribution;
applying an attention mask to filter the symptom answers and the test results for obtaining an attention input; and
generating another explainable description about the predicted disease list according to the attention input.

10. A control method, comprising:
collecting a symptom input status and a test result status, the symptom input status comprising a plurality of symptom answers, the test result status comprising a plurality of test results;
utilizing a neural network to generate a predicted disease distribution according to the symptom input status and the test result status;
generating a predicted disease list according to the predicted disease distribution;
applying an attention mask to filter the symptom answers and the test results for obtaining an attention input; and
generating an explainable description about the predicted disease list according to the attention input.

11. The control method as claimed in claim 10, wherein the explainable description corresponds to at least one of the symptom answers passing the attention mask or at least one of the test results passing the attention mask.

12. The control method as claimed in claim 10, wherein the attention mask is generated by an attention module according to the symptom input status and the test result status.

13. The control method as claimed in claim 12, wherein the attention module is trained to generate the attention mask in reference with known medical records.

14. A medical system, comprising:
an interface, configured for receiving a symptom input status and a test result status, the symptom input status comprising a plurality of symptom answers, the test result status comprising a plurality of test results; and
a processor coupled with the interface;
wherein in a test suggestion phase, the processor utilizes a neural network to generate a test suggestion, a predicted test result distribution and a predicted disease distribution according to the symptom input status and the test result status, the test suggestion comprising a candidate test, the processor estimates a plurality of information gains of the candidate test relative to a plurality of diseases according to the predicted test result distribution and the predicted disease distribution, and the processor generating a first explainable description about the test suggestion according to the information gains of the candidate test.

15. The medical system as claimed in claim 14, wherein the first explainable description indicates a disease list that the candidate test is capable to distinguish according to the information gains, the neural network is trained by the processor to generate the test suggestion, the predicted test result distribution and the predicted disease distribution in reference with known medical records.

16. The medical system as claimed in claim 14, wherein the processor is further configured to:
utilize the neural network to generate a symptom query;
collect a symptom answer corresponding to the symptom query; and
update the symptom input status according to the symptom answer.

17. The medical system as claimed in claim 14, wherein in a disease prediction phase, the processor generates a predicted disease list according to the predicted disease distribution, the processor applies an attention mask to filter the symptom answers and the test results for obtaining an attention input, the processor generates a second explainable description about the predicted disease list according to the attention input.

18. The medical system as claimed in claim 17, wherein the second explainable description indicates at least one of the symptom answers passing the attention mask or at least one of the test results passing the attention mask.

19. The medical system as claimed in claim 17, further comprising:
an attention module, executed by the processor for generating the attention mask according to the symptom input status and the test result status.

20. The medical system as claimed in claim 19, wherein the attention module is trained to generate the attention mask in reference with known medical records.
